(19) United States
(12) Patent Application Publication
(10) Pub. No.: US 2008/0294160 A1
(43) Pub. Date: Nov. 27, 2008

(54) RF endoscopic electrosurgical instrument

(76) Inventors: Jon C. Garito, Oceanside, NY (US); Alan G. Ellman, Oceanside, NY (US)

Correspondence Address: JACK OISHER, 200 HIGH POINT DRIVE, SUITE PH2, HARTSDALE, NY, 10530, US

(21) Appl. No.: 11/804,981

(22) Filed: May 22, 2007

(51) Int. Cl. A61B 18/18

(52) U.S. Cl. 606/46

(57) ABSTRACT

A unipolar electrosurgical electrode or probe that is configured for use in MIS electrosurgical procedures. The electrosurgical electrode comprises an elongated tubular member configured to cooperate with the small cannula of a mini-endoscope to reach interior tissue. The active end comprises a pair of jaws that can be normally closed and operative by the surgeon to open and close down on and grasp tissue to excise same. When energized, a unipolar discharge is generated at the working end of the electrode.

This invention relates to an electrosurgical probe for medically treating tissue lesions by minimally invasive surgery (MIS) or similar procedures.

### BACKGROUND OF THE INVENTION

Our prior patent, No. 7,160,295, whose contents are incorporated herein by reference, describes an improved electrosurgical probe for endoscopic endonasal and other procedures using a standard operating room working channel fiberoptic scope or endoscope. It employs a very long, thin, flexible, insulated, monopolar wire electrode, so thin and flexible that it can be used with a miniature or micro-sized endoscope combining imaging optics and an instrument. The endoscope may be sufficiently flexible so as to follow the contours of the patient's anatomy, and the wire electrode is sufficiently flexible so as to follow the contours of the working channel of the endoscope. A particular application of importance is the treatment of benign lesions of the larynx, especially of the vocal cords, through flexible endoscopy.

There is also a need in the art for rigid devices to simplify the treatment by MIS of tissues which cannot be easily reached by many instruments, such as epidural scar tissue, adhesions and other pathology, and spinal diseases such as intradiscal shrinkage or ablation.

### SUMMARY OF THE INVENTION

An object of the invention is an improved electrosurgical probe for treating tissue.

Another object of the invention is an improved electrosurgical probe for treating tissue that can use a standard operating room working channel fiberoptic scope or endoscope.

Still another object of the invention is an improved electrosurgical probe that can be used with flexible steerable or with rigid endoscopes.

Still another object of the invention is an improved radio-frequency (RF) electrosurgical forceps for treating tissue that has in the past been treatable with difficulty and with troublesome bleeding on occasions.

In accordance with a feature of the invention, an electrosurgical probe comprises an elongated tubular member having at a proximal end a handle and at a distal end a pair of electrically-conductive jaws that can be closed under control of a surgeon operating the handle to grasp tissue. The elongated tubular member can be made sufficiently flexible that it can be used with a miniature or micro-sized endoscope combining imaging optics and an instrument channel with an overall diameter below about 3 mm or with a flexible steerable endoscope. It can also be made sufficiently stiff that it can be used with a rigid endoscope. Because of its capability of use with a miniature or micro-sized endoscope in a standard operating environment, hospital or office, it allows a surgeon to conduct a surgical procedure with improved visualization of the surgical site.

In a preferred embodiment, the probe is monopolar with both jaws connected to the electrosurgical source of electrosurgical currents. The jaws when closed form a kind of clam-shaped or boat-shaped body with the facing peripheral edges having a relatively sharp edge for tissue cutting and to focus the RF currents at the edges when they grip tissue to be removed. Preferably, one of the jaws has a sharp tooth facing the other jaw. The tooth functions to grasp tissue, and/or to hold the tissue in case of movements, and/or to tightly hold the tissue to be excised to prevent its being inadvertently dropped when excised during the procedure.

By “proximal” is meant the end closest to the connector, and by “distal” is meant the end furthest from the connector.

The construction of the invention will provide important benefits for all MIS arthroscopic or endoscopic procedures and in many cases enables the efficient delivery of radiofrequency (RF) energy technology for controlled precise tissue cutting, absorption and other tissue effects and in a safe manner. It is cost effective and considerably less expensive than other surgical modalities such as lasers where the novel electrode configuration may be of importance, as well as for general electrosurgical procedures where the volumetric reduction of tissue or ablation of tissue that is hard to reach with the known electrodes is desirable. Examples of particular procedures for which the electrosurgical electrode of the invention is particularly suitable are spinal disc ablation surgery through a cannula for treatment of herniated or bulging discs or during spinal fixation surgery, endoscopic gastroenterological surgery, and endoscopic surgery.

The various features of novelty which characterize the invention are pointed out with particularity in the claims annexed to and forming a part of this disclosure. For a better understanding of the invention, its operating advantages and specific objects attained by its use, reference should be had to the accompanying drawings and descriptive matter in which there are illustrated and described the preferred embodiments of the invention, like reference numerals designating the same or similar elements.

### BRIEF DESCRIPTION OF THE DRAWINGS

In the drawings:

FIG. 1 is a plan, partly cross-sectional view of one form of an electrosurgical probe according to the invention along the line 1-1 of FIG. 2 shown with normally-closed jaws;

FIG. 2 is a top view of the electrosurgical probe shown in FIG. 1;

FIG. 3 is a side and partially cross-sectional view of the electrosurgical probe shown in FIG. 1 with the jaws open and shown connected by a cable to electrosurgical apparatus;

FIG. 4 is an enlarged side view of the jaws of the working end in open position of the probe of FIG. 3;

FIG. 5 is an enlarged perspective view of the jaws of the working end in open position of the probe of FIG. 3.

FIG. 6 is an enlarged perspective view of the jaws of the working end in closed position of the probe of FIG. 1.

FIG. 7 is a schematic view illustrating the working end of the probe extending through an endoscope;

FIG. 8 is a plan, partly cross-sectional view of another form of an electrosurgical probe according to the invention;

FIG. 9 is an enlarged perspective cross-sectional view of the handle end of the probe of FIG. 8;

FIG. 10 is an enlarged side view of the jaws of the working end in open position of the probe of FIG. 8;

FIG. 11 is an enlarged side view of the jaws of the working end in closed position of the probe of FIG. 8.

### DETAILED DESCRIPTION OF PREFERRED EMBODIMENTS

The reader is directed to the referenced prior patent for a more detailed description of electrosurgical procedures and principles of operation which will assist in understanding the invention described in the present application.

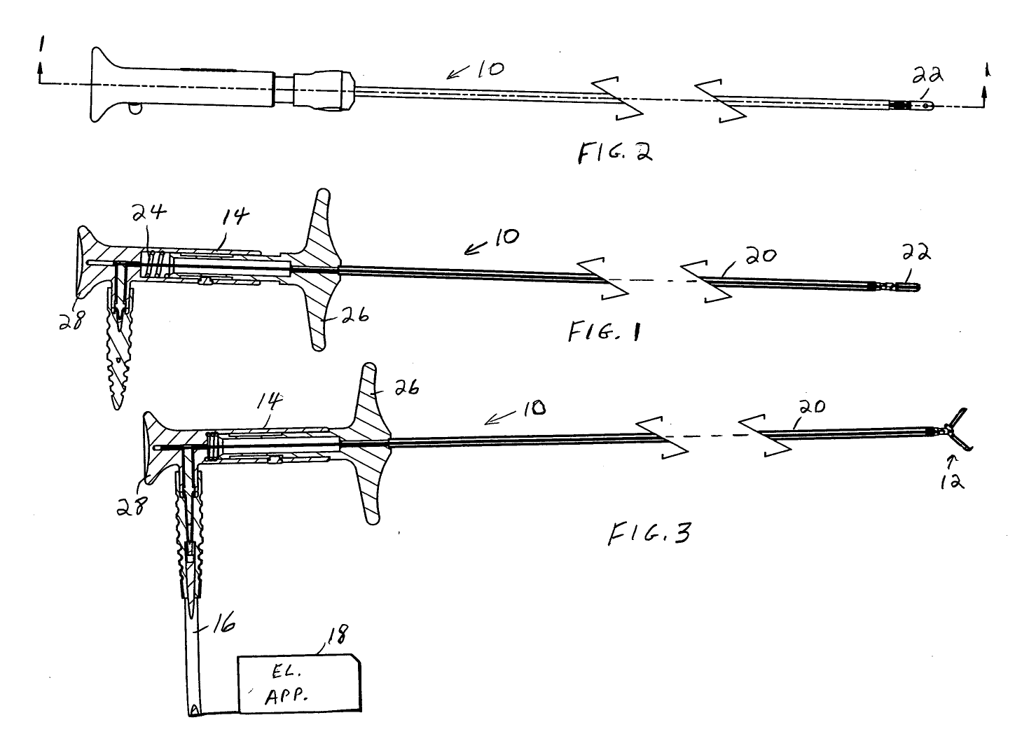

In the present application, FIG. 7 is a generally schematic view of the end of an endoscope 5 with two working channels. The first 7 is for receiving one form of electrosurgical probe electrode 10 in accordance with the invention. The second, which is underneath the first channel and thus invisible, comprises the usual scope by which the surgeon views the surgical site in a MIS procedure. Only the working end 12 of the probe 10 is shown. The entire instrument 10 is illustrated in FIGS. 1 and 3, and comprises a handle 14, to which is connected a cable 16 (FIG. 3) and in turn to electrosurgical apparatus 18 schematically illustrated. From the handle 14 extends an insulated tubular member 20 terminating at the right side of the drawing in the working end 12 comprising a pair of bare electrically-conductive, for example, of metal, jaws 22. The cable 16 connects internally to the electrically-conductive jaws 22 so that when the electrosurgical apparatus is activated, electrosurgical currents flow to the jaws 22.

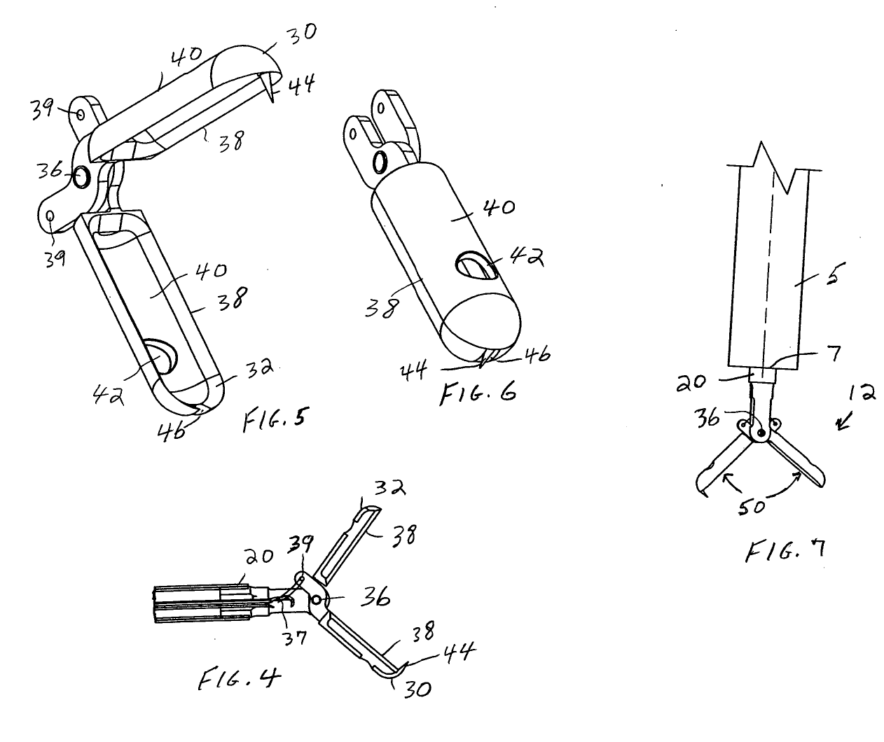

As is evident from FIGS. 3 and 4, the jaws 22 comprise a toothed jaw 30 and a matching untoothed jaw 32, without a tooth, pivoted together at 36 on the end of the tubular member 20. Each jaw has a similar shape. In the embodiment illustrated (see FIGS. 5 and 6), each jaw has a boat-like shape with a raised perimeter 38 about a mostly solid back surface 40, except for one opening 42 that functions as a cleaning entrance for cleaning or sterilizing fluids. The perimeters 38 are ground to form sharpened edges. The two jaws 30, 32 are so similar that when closed as in FIG. 6 their sharpened edges 38 meet. When the jaws are machine ground, the perimeter edges are left sharp. These sharp edges not only increase the efficiency of a cold cut of the tissue (with no RF energy) when the sharpened edges meet on the tissue, but also, when RF energy is applied, the sharpened edges focus the energy to these sharp edges creating a more efficient hot cut (with RF energy). The sharpened edges thus serve to focus the electrosurgical currents at the edges 38 which come in contact with the tissue to be excised. A typical length of the boat-like body for larynx surgery is about 0.2-0.3 inches. The width is about 0.06-0.08 inches. The jaws 30, 32 can even be reduced in length to about 0.1 inches, retaining the same width so that the jaws when closed form a kind of clam-shaped body. The tiny size of the jaws is what makes them so effective for removing small lesions from, for example, vocal cords. The upper jaw 30 (in FIG. 6) differs from the lower jaw 32 by having a tooth 44 at its distal end, the tooth 44 projecting downward toward the lower jaw and engaging a recess 46 in the lower jaw in the closed jaw position. The tooth extends about 0.04 inches below its perimeter, and functions to grip the tissue and hold the tissue in case of movement and when excised by the closing jaws.

The jaws 22 in this example are spring loaded 24 to a closed position, which position they occupy when they are extended through the instrument channel. The surgeon holds the handle with two fingers at a cross piece 26, and with his thumb on the handle end 28. When the surgeon squeezes the handle end, the jaws assume their open position (shown in FIG. 3). This is achieved by means of a pair of internal wires, fixed at the handle end 28, which are pushed forward when the handle is squeezed pushing forward two internal wire rods 37 whose distal ends are fixed, respectively, to the pivot ends 39 of each of the jaws. In the unsqueezed condition with the jaws closed, the jaw ends can be inserted through the working channel and the extended jaws will be maintained in their closed position until the surgeon squeezes the handle whereupon the jaws open to open position. The surgeon then advances the open jaws to the surgical site and tissue to be excised, and releases the pressure on the handle 14, the spring 24 pressure closing the jaws on the tissue while the surgeon can activate the electrosurgical apparatus 18 for a hot cut.

A typical application is a procedure for the removal of polyps or the excision of benign lesions of the larynx, and especially of the vocal cords. As one preferred procedure, a flexible endoscope is introduced through the nose. When it reaches the vestibule of the larynx, the electrosurgical probe of the invention with the dimensions of a microfiber is placed into the working channel, which may be for example of 4.8 mm outside diameter and 2.2 mm inside diameter. A typical length of the tubular member 20 is about 7-10 inches with a diameter of about 2 mm. Advancing, withdrawing, and rotating the endoscope 5 permits manipulation of the active electrode end 12 in three dimensions. For large lesions, the jaws 22 are applied to the pedicle while applying cut- or cut-coagulation waveform RF electrosurgical currents from the electrosurgical apparatus 18. A power of 5 (as displayed on the instrument) can be used and varied according to the tissue response. The pedicle is cut gradually as the instrument is advanced through the endoscope until a slim pedicle remains, which can be removed while held by the jaw tooth 44 or by using conventional flexible forceps. The jaws 22 provide tactile grabbing of polyps, nodules and granulomas on the tissue. The small tooth 44 on the tip of the instrument will catch or grab the tissue which is often moving due to airway breathing. Additionally the tooth holds the tissue in place and avoids movement during the extirpation or cutting-coagulation. The jaw's configuration of straight and lengthened design with sharpened edges allows a precise cut as the RF energy flows to the metal jaws. The cut-coag RF waveforms then provide a sealing of any bleeders with minimal heat spread. The jaw tooth is important to prevent the excised tissue from falling into the lower airway. FIG. 7 demonstrates that for a typical endoscope with an 85° field of view, the jaws 12 can be opened to a similar angle indicated by reference numeral 50.

The electrosurgical instrument of the invention is important especially for nodules of the volcal cords, polyps, and granulomas. It has the important advantages of providing a clean cut and hemostasis simultaneously. Moreover, the jaws with its tooth are important because they can grip a vocal cord tumor for excision despite abnormal movement or spasm of the cords.

The application of a 4 MHz RF in surgical procedures of the larynx causes minimal dispersion of heat beyond the tip of the electrode, allowing precise cutting and coagulation thus minimizing bleeding.

The tubular member 20 and the jaws 22 are preferably made of stainless steel though other electrically conductive metals can be used such as brass. The exposed surface of the tubular member is coated with an electrically-insulated coating. Only the jaws are bare.

A further advantage is obtained when the electrode of the invention is used with electrosurgical apparatus capable of generating RF electrosurgical currents at frequencies of about 4 MHz. The monopolar electrode jaws enables the efficient delivery of RF energy and is uniquely suited for procedures requiring controlled precise tissue excision. It offers the further advantage that it delivers lower tissue temperature profiles. Moreover, it allows more easily the extension of RF electrosurgical currents to minimal and micro invasive surgical procedures. Minimal and micro surgical procedures typically result in reduced pain and scarring, shorter recovery time and increased effectiveness compared to traditional surgical procedures. An example of suitable electrosurgical apparatus is the Model SURGITRON Dual-Frequency electrosurgical unit manufactured by and available from Ellman International, Inc. of Oceanside, N.Y.

Once the surgeon has positioned the working end 12 of the electrode with respect to the tissue to be operated on, he or she then activates the electrosurgical apparatus 18 causing a discharge of unipolar currents between a ground plate (not shown) and the bare jaws 22 capable of causing excision or ablation or shrinkage of tissue or cauterization of a blood vessel in the usual way. As with the embodiments of the prior patents, the insulating coating on the tubular member 20 will prevent accidental touching of any conductive members or patient tissue by the electrode sides, so that the unipolar discharge is localized to the region surrounding the working end 12.

The electrosurgical probe of the invention, because it combines grasping, cutting, coagulation, and soft tissue ablation, in the form of a flexible tubular member has, apart from larynx procedures, many other applications in the following endoscopic minimally invasive surgical procedures: endonasal; skull base; endosinus and transphenoidal surgery; tracheobronchial procedures; subglottic or tracheal stenosis and lesions; quick radial incisions with low tracheal perforation risk; transoral, laryngeal and orapharyngeal surgery. It can be configured to easily reach the sublaottis and the trachea, and cut and ablate airway lesions. It can be used in either flexible or rigid tip delivery.

In the embodiment of FIG. 1, the jaws are spring loaded 24 to a normally-closed position. Alternatively, they can be spring-loaded to the normally-open position, in which case the surgeon would squeeze the jaws closed to pass through the endoscope and then release the pressure for the jaw to open and again squeeze to close the jaws on tissue at the surgical site.

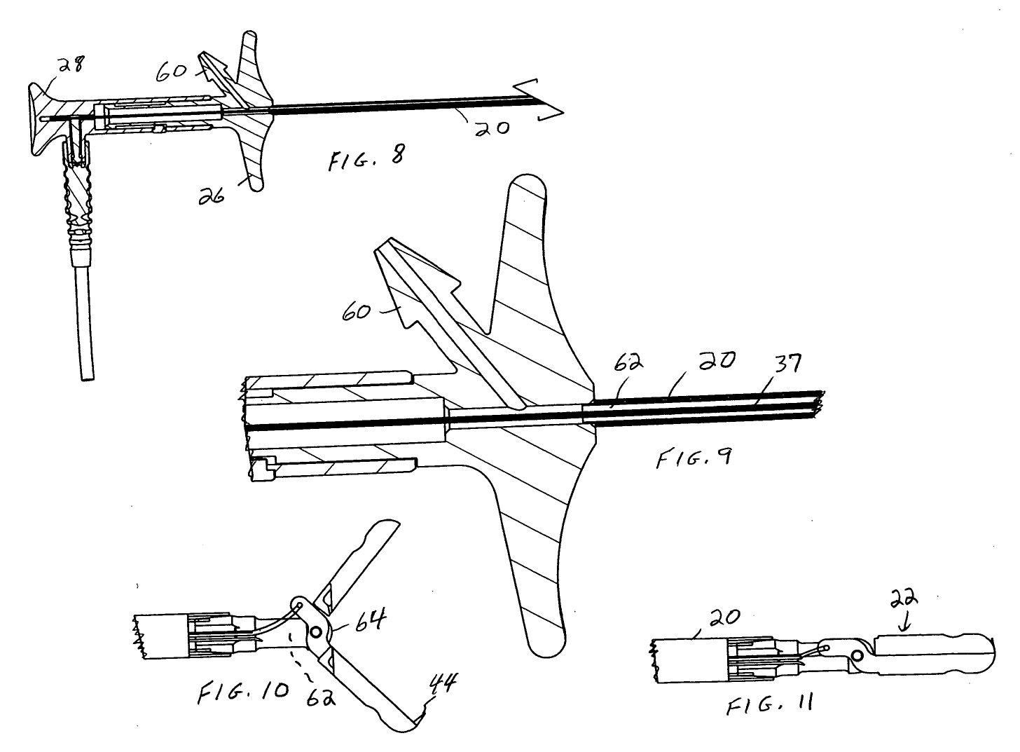

FIGS. 8 and 9 show another embodiment in which the spring is omitted. In this case, the device becomes hand actuated both ways and the surgeon pushes in or out the end 28 relative to the cross piece 26 to place the jaws in the desired position. This embodiment also shows a variant wherein suction is provided at the open jaw end to remove fluid and loose tissue. A suction source (not shown) is connected via a fitting 60 to the hollow tube 20. This is more clearly shown in FIG. 9. The suction fitting 60 is connected via its internal conduit to the interior 62 of the tubular member 20. At the distal end, when the jaws are open, shown in FIG. 10, the tubular member 20 terminates in an open port 64. When the jaws close, shown in FIG. 11, the port 64 is closed and the suction decreases. The presence of the suction at the jaws reduces amount of fluid in the surgical site and improves operative visibility.

In this description, by “elongated” or “longitudinal” is meant parallel to the long axis of the electrode (horizontal in FIG. 1).

While the invention has been described in connection with preferred embodiments, it will be understood that modifications thereof within the principles outlined above will be evident to those skilled in the art and thus the invention is not limited to the preferred embodiments but is intended to encompass such modifications.

What is claimed is:

1. An electrosurgical probe for treating tissue, comprising:
(a) an elongated tubular member having a major longitudinal axis and having a proximal first end and a distal second end and being configured to pass through a working channel of an endoscope,
(b) the tubular member terminating at the distal end in an electrically-conductive grasping member,
(c) means at the proximal end connected to the grasping member for causing the grasping member to grasp and ungrasp tissue,
(d) means at the proximal end for supplying electrosurgical currents to the grasping member.

2. The electrosurgical probe as claimed in claim 1, wherein the grasping member comprises a pair of pivoted jaws capable of assuming an open position and a closed position.

3. The electrosurgical probe as claimed in claim 1, wherein the grasping member is configured such that the pivoted jaws are sprung into a normally closed position.

4. The electrosurgical probe as claimed in claim 3, wherein the pivoted jaws are normally closed and are opened by the means for causing the grasping member to grasp and ungrasp tissue.

5. The electrosurgical probe as claimed in claim 2, wherein the jaws when closed form a boat-shaped or clam-shaped body.

6. The electrosurgical probe as claimed in claim 5, wherein the jaws each comprise a back surrounded on three sides by a perimetrical sharp edge.

7. The electrosurgical probe as claimed in claim 6, wherein one of the jaws comprises a tooth located at the point furthest from the proximal end.

8. The electrosurgical probe as claimed in claim 6, wherein the tooth extends toward the other jaw.

9. The electrosurgical probe as claimed in claim 7, wherein one of the jaws comprises a recess for receiving the tooth located at the point furthest from the proximal end.

10. The electrosurgical probe as claimed in claim 6, wherein the jaws each comprise a cleaning opening in the back.

11. The electrosurgical probe as claimed in claim 1, wherein the outside diameter of the tubular member is less than about 3 mm.

12. The electrosurgical probe as claimed in claim 7, wherein the length of the jaws is about 0.1-0.3 inches.

13. The electrosurgical probe as claimed in claim 7, wherein the width of the jaws is about 0.06-0.08 inches.

14. A surgical procedure using a mini-endoscope having a channel of about 4 mm or less, comprising the steps:
A) positioning the viewing end of the endoscope at a surgical site,
B) inserting into the channel an electrosurgical electrode probe comprising:
(a) an elongated tubular member having a major longitudinal axis and having a proximal first end and a distal second end and being configured to pass through the channel of the endoscope,
(b) the tubular member terminating at the distal end in an electrically-conductive grasping member,
(c) means at the proximal end connected to the grasping member for causing the grasping member to grasp and ungrasp tissue,
(d) means at the proximal end for supplying electrosurgical currents to the grasping member.
(e) the grasping member comprising a pair of pivoted jaws capable of assuming a closed position and an open position,
C) supplying electrosurgical currents to the proximal end to reach and activate the pivoted jaws,
(D) closing the jaws on tissue at the surgical site to excise the tissue.

15. The surgical procedure of claim 14, wherein the supplied electrosurgical currents are at a frequency of about 4 MHz.

16. The surgical procedure of claim 15, wherein the procedure is removing tissue from the larynx.

17. The surgical procedure of claim 15, wherein the procedure is an endonasal procedure.

18. The surgical procedure of claim 15, wherein the procedure is a spinal procedure.
